(19) United States
(12) Patent Application Publication
(10) Pub. No.: US 2024/0017031 A1
(43) Pub. Date: Jan. 18, 2024

(54) ADJUSTABLE FACEPLATE FOR RESPIRATORY MASKS

(71) Applicant: Boaz Barry Groman, Boca Raton, FL (US)

(72) Inventor: Boaz Barry Groman, Boca Raton, FL (US)

(21) Appl. No.: 18/235,809

(22) Filed: Aug. 19, 2023

Related U.S. Application Data
(63) Continuation-in-part of application No. 18/225,175, filed on Jul. 24, 2023.
Application No. 18/225,175 is a continuation-in-part of application No. 17/734,133, filed on May 2, 2022.
Application No. 17/734,133 is a continuation-in-part of application No. 17/734,133, filed on May 2, 2022.
(60) Provisional application No. 63/371,872, filed on Aug. 19, 2022.
(60) Provisional application No. 63/182,937, filed on May 2, 2021.

(51) Int. Cl. A61M 16/06; A61M 11/00; A61M 16/10

(52) U.S. Cl. CPC A61M 16/0605; A61M 11/00; A61M 16/0683; A61M 16/105

(57) ABSTRACT

A relatively rigid, adjustable faceplate fitted over and working in conjunction with a relatively flexible respiratory facemask. The faceplate pushes (urges) the facemask against a user's (patient's) facial features to assure a better fit of the facemask to the patient's face. Straps are disposed on the faceplate, rather than on the facemask. The faceplate may provide or comprise: filter protection from user contact, strap locking features, cutouts for accommodating stick-on filters mounted to the facemask, and a nebulizer cutout. An upper portion of the faceplate is provided with a slider to allow the faceplate to achieve an effective fit when used with different size (adult, child, pediatric) facemasks by adjusting the distance between a nose pressure point and a chin pressure point of the faceplate.

### CROSS-REFERENCE TO RELATED APPLICATIONS

This is a nonprovisional filing of U.S. 63/371,872 filed 19 Aug. 2022.

This is a continuation-in-part of U.S. Ser. No. 18/225,175 filed 24 Jul. 2023, which is a continuation-in-part of U.S. Ser. No. 17/734,133 filed 2 May 2022, which is a nonprovisional filing of U.S. 63/182,937 filed 2 May 2021.

This is a continuation-in-part of U.S. Ser. No. 17/734,133 filed 2 May 2022, which is a nonprovisional filing of U.S. 63/182,937 filed 2 May 2021.

The aforementioned Ser. No. 18/225,175 filed 24 Jul. 2023 has not yet been published

The aforementioned Ser. No. 17/734,133 filed 2 May 2022 has been published as US 20220347403 (2022-11-03)

### FIELD OF THE INVENTION

This invention relates to medical facemasks, more particularly to respiratory facemasks and, more particularly to securing respiratory facemasks to the face of a patient.

This invention may also relate to respiratory facemasks incorporating means for filtering of air being breathed by a patient, particularly air which is exhaled by the patient which may contain undesirable aerosols and/or particulate matter (including suspensions).

This invention may also relate to respiratory facemasks incorporating means for providing low pressure oxygen and/or nebulizing treatments to a patient.

### BACKGROUND

In the 2020 Coronavirus pandemic, treatment options for patients with shortness of breath were limited due to the fear that first line and hospital personnel would be exposed to aerosolized viral particles expelled by patients into the environment.

Typically, for shortness of breath, the first line of treatment is oxygen. However, it soon became apparent, when treating patients with SARS-CoV-2, that oxygen via a nasal cannula at greater than six liters per minute, aerosolized viral particles and exposed personnel caring for the patient. In addition, patients with asthma and chronic obstructive pulmonary disease could not be treated with nebulized medications, the mainstay of these diseases, for the same reason.

Due to recent infections, ERs have stopped providing breathing treatments for all illnesses (asthma, heart, OCPD, Etc.) due to aeration concerns. Instead, many more patients are being intubated so ventilated air can be controlled. Intubation is a substantially more invasive, risky, and costly procedure.

The following publications may have some relevance to the present invention:
U.S. Pat. No. 6,659,102 (2003-12-09; Sico)
U.S. Pat. No. 6,854,464 (2005-02-15; Mukaiyama et al)
U.S. Pat. No. 7,559,323 (2009-07-14; Hacke et al; Respan Products Inc.)
U.S. Pat. No. 8,342,179 (2013-01-01; Hacke et al)
U.S. Pat. No. 8,464,715 (2013-06-18; Flynn, Sr.)
U.S. Pat. No. 8,534,280 (2013-09-17; Dhuper et al)
U.S. Ser. No. 10/335,569 (2019-07-02; Beard et al)
U.S. Ser. No. 10/576,313 (2020-03-03; Shigematsu et al)
D753816 (2016-04-12; Beard)
US 20050028811 (2005-02-10; Nelson et al)
US 20130125896 (2013-05-23; Dwyer et al)
US 20160184548 (2016-06-30; Wallnewitz et al)
US 20210330912 (2021-10-28; Groman et al)
US 20220347403 (2022-11-03; Groman)

### SUMMARY

It is an object of the invention, in some of the embodiments disclosed herein, to provide improvements to using medical facemasks. The present invention may be particularly applicable to use with respiratory facemasks.

According to the invention, generally, a relatively rigid, adjustable faceplate may be fitted over and work in conjunction with a relatively flexible respiratory facemask (mask). The faceplate pushes (urges) the mask against a user's facial features to assure a better fit. Straps are disposed on the faceplate, rather than on the mask. The faceplate may provide or comprise: filter protection from user contact, strap locking features, and a nebulizer cutout. An upper portion of the faceplate is provided with a slider to allow the faceplate to achieve an effective fit when used with different size (adult, child, pediatric) facemasks by adjusting the distance between a nose pressure point and a chin pressure point of the faceplate.

A “bridge” or a “decorative shield” may be incorporated into the faceplate, extending generally horizontally across the faceplate, from left-hand side to right-hand side thereof, approximately midway between the top and bottom thereof, and may be integrally formed with the faceplate. The purpose(s) of the decorative shield may be to enhance rigidity of the faceplate, protect filters mounted to the facemask from contact (contamination), to make faceplate more friendly for kids (they make masks with characters on them just for that purpose), to add grasping supports, or to enhance perceived value, including combinations thereof. The fixed (non-adjustable) faceplate, without the “bridge” or a “decorative shield” is prior art.

According to an embodiment of the invention, an adjustable faceplate for securing a respiratory facemask to the face of a patient may comprise: a structure having an opening having a height “H” and a width “F”, and a peripheral portion conforming to a corresponding peripheral portion of the facemask, and suitable to be disposed over the facemask when the facemask is on the patient's face; and at least one strap extending from the structure for securing the faceplate to the patient's face, thereby negating a need for a straps extending from the facemask; a slider disposed at a top portion of the faceplate for providing a nose pressure point, wherein the a distance between the nose pressure point and a chin pressure point at a bottom portion of the faceplate is adjustable by moving the slider so that the faceplate can perform well with masks of different sizes for adult, child, and pediatric patients.

The faceplate may be more rigid than the facemask. The structure may formed of a material that is thicker and more rigid than the material of the facemask. The faceplate spreads forces from the strap(s) more evenly around the peripheral portion of the facemask, thereby enhancing a seal between the facemask and the patient's face. The faceplate may have a cutout disposed at a bottom portion thereof to accommodate mounting a nebulizer device. The faceplate may be of sufficient width to accommodate filters attached to facemask.

According to an embodiment of the invention, an adjustable faceplate, for use with respiratory masks may comprise: an adjustable nose support element comprising: a moveable “slider” element extending from a top portion of the faceplate into a nose opening of the faceplate; and features on a top portion of the faceplate for grasping the slider in a manner that it may be moved up and down to accommodate patients having different size faces. The slider element may be selectively repositionable to adjust the “fit” of the faceplate.

The slider element may be is provided with grooves or ridges on a surface thereof, and the corresponding features on the top portion of the faceplate may comprise grooves or ridges for interacting (mating) with the grooves or ridges on the slider. The grooves or ridges on the slider and the top portion of the faceplate allow the slider to be moved up or down to accommodate masks of different sizes and to retain the slider in place when the faceplate is installed on the patient. The slider allows the spacing (distance between a nose pressure point and a chin pressure point of the adjustable faceplate) to be modified (adjusted) according to the size of the patient's face (and facemask being used), so that the adjustable faceplate can exert appropriate pressure upon the face mask to ensure (improve) sealing of the facemask on (to) the patient's face.

In use, the faceplate pushes (urges) the mask against a wearer's facial features, particularly at a nose pressure point and a chin pressure point.

According to an embodiment of the invention, a method of mounting an adjustable facemask onto the face of a patient may comprise: applying (mounting) a medical (such as respiratory) facemask onto the patient's face; and securing the facemask in place on the patient's face with a faceplate. The faceplate includes a strap, thereby obviating the need to the facemask to have a strap (although the facemask may also have a strap). The faceplate provides a more secure mounting of the facemask to the patient's face

The height of a nose opening (distance “H” between a nose pressure point and a chin pressure point) in the faceplate may be adjusted to accommodate different size facemasks.

The faceplate is arranged (with cutouts) extending from the nose opening to accommodate filters which may be attached (mounted) to the facemask.

A nebulizer may be mounted to the faceplate.

The invention may work well in conjunction with the respiratory facemask and filter devices disclosed in US 20220347403.

Other objects, features and advantages of the invention(s) disclosed herein may become apparent in light of the following illustrations and descriptions thereof.

### BRIEF DESCRIPTION OF THE DRAWINGS

Reference will be made in detail to embodiments of the disclosure, non-limiting examples of which may be illustrated in the accompanying drawing figures (FIGS.). The figures may generally be in the form of diagrams. Some elements in the figures may be stylized, simplified or exaggerated, others may be omitted, for illustrative clarity.

Although the invention is generally described in the context of various exemplary embodiments, it should be understood that it is not intended to limit the invention to these particular embodiments, and individual features of various embodiments may be combined with one another. Any text (legends, notes, reference numerals and the like) appearing on the drawings are incorporated by reference herein.

FIG. 1 is a diagram of a non-adjustable face plate of the prior art, such as shown in FIG. 5 of US 20210330912 (with some alterations).

FIG. 2 is an illustration of an adjustable face plate (or faceplate), according to an embodiment of the invention.

FIG. 2A is an illustration of a portion of the adjustable face plate (or faceplate) of FIG. 2, according to an embodiment of the invention. The view is an external view.

FIG. 2B is an illustration of a portion of the adjustable face plate (or faceplate) of FIG. 2, according to an embodiment of the invention. The view is an internal view.

FIG. 2C is a diagram (cross-sectional view) of the adjustable face plate (or faceplate) of FIG. 2, according to an embodiment of the invention.

### DESCRIPTION

US 20210330912 (2021-10-28; Groman et al.) discloses several innovations that make possible the conversion of vented nebulizer and oxygen masks into filtered masks. However, the embodiments disclosed in US 20210330912 have the following shortcomings:

Because a minimum filter size is required to permit the needed air flow, plus allowance for degradation in filter effectiveness due to nebulizer aerosol clogging the filters, the largest possible mask size is utilized. Smaller size masks do not have sufficient ‘real estate’ or ‘flat surfaces’ to directly weld the required filter size onto the mask per the embodiments in US 20210330912.

Because a large mask is utilized, the fit for small-face individuals results in gaps around facial features, most predominantly the chin and nose bridge. Therefore, real-world products based on the embodiments in US 20210330912— SafetyNeb™ and SafetyO2™—utilize nose and chin pads to close those gaps. The cost of fabricating and mounting the pads adds substantial cost to the real world products and the added pads are still susceptible to leakage in some individuals due to varying facial features. SafetyNeb™ and SafetyO2 are the marketed products that were developed out of US 20210330912 and utilize the single (fixed) size faceplate. www.Aerosoless.com

These products are not usable on pediatric patients because the pads are not sufficient to overcome these gaps in children.

The faceplate device of US 20210330912 is fixed in size to optimize the pressing of the mask onto the facial features. This faceplate device would only work with the mask size selected for the real-world products and would not function properly on any other mask sizes.

US 20220347403 (2022-11-03; Groman), directed to stick-on filters, allows for the conversion of any vented mask into a filtered mask. US 20220347403 solves the problem of real-estate restriction and curvature issues by mounting and conforming to the mask vents without filter size restrictions. Therefore, any mask size may be utilized and converted into a filtered mask, even pediatric masks. Additionally, various size filters could be made available to facilitate various procedure types and durations.

The ability to utilize smaller mask sizes eliminates the ‘stop gap’ measure of utilizing the nose and chin pads. So with the Exhalation Filtration Device described in US 20220347403, healthcare providers can select a mask size suitable for each individual patient and expediently convert the selected mask into a filtered mask. Since masks of various sizes may be utilized, the faceplate of the US 20210330912 would not function properly and in the real-world application require many fixed-size faceplates that fit each brand and size of masks on the market.

To solve this issue, and to work in conjunction with the US 20220347403 Exhalation Filtration Device, an adjustable faceplate is disclosed herein that can accommodate any mask size or make. The combination of the US 20220347403 Exhalation Filtration Device and an adjustable faceplate provides the ultimate flexibility to healthcare providers, since it allows them to select the proper size masks, reduces product cost, and allows for the reusability of masks on same patient over an extended period of treatments.

### APPENDICES

The following Appendices were included with the filing of US 20210330912:
Appendix 1: Face Mask Filter Retrofit Prototype Description
Appendix 2: Face Mask Filter Retrofit—Alternative Embodiments
Appendix 3: Current And Improved Nebulizer Methods
Appendix 4: Face Plate Embodiment
Appendix 5: Spacer
Appendix 6: Integrating (Welding) B/V Filters onto a Standard Nebulizer Mask

The above-referenced Appendix 4 is appended hereto as Appendix A.

Appendix 7 was included in the filing of 63/371,872 filed 19 Aug. 2022, and is appended hereto as Appendix B.

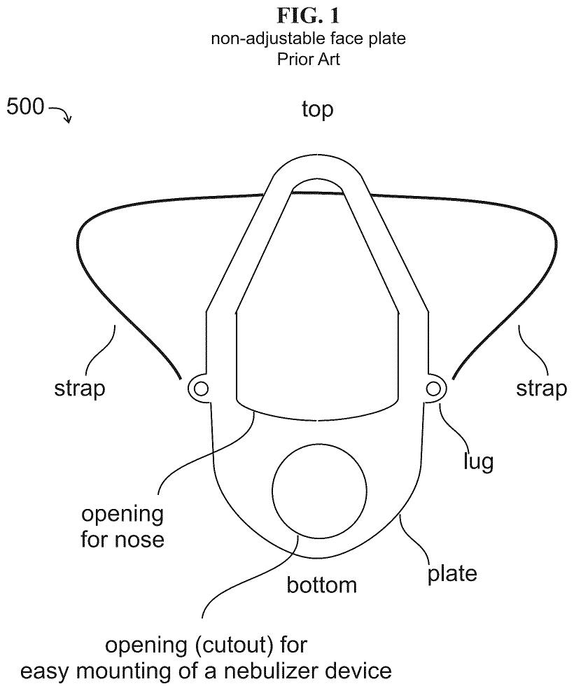

FIG. 5 of US 20210330912 shows a faceplate, and is presented herein as FIG. 1 (with some modifications).

The opening in the lower portion of the faceplate had previously been referred to as an opening for mouth (or mouth opening). More appropriately, this opening is a cutout for mounting a nebulizer device.

As set forth in US 20210330912,

FIG. 5 is a diagram (plan view) of a face plate for use with a face mask, according to an embodiment of the invention.

As described US 20210330912, with respect to Appendix 4 therein: Face Plate Embodiment

It was determined that low cost masks leak around the facial features due to their material and cheap manufacturing.

The filters may be attached to the masks themselves (integral style) instead of making plastic cases to house the filters.

A “face plate” is provided that pushes the masks against the facial features to assure a much better fit, and also has some additional features, as may be evident from Appendix 4 (Appendix A).

Page 1 Standard Mask for Nebulizing and Oxygen Treatments (Prior Art)

This page shows some views (Front/Outer, Side, Back/Inner) of a standard mask for nebulizing and oxygen treatments. Note that there open ports in the mask to permit patient breathing.

This invention may comprise some modifications to a standard mask (see, e.g., page 2), and also the addition of a face plate (see, e.g., page 3).

Page 2 Modifications to the Standard Mask to Reduce Aerosolization

This page shows a Back/Inner View of a modified mask. The following features are highlighted.

Added Nose Cushion to Increase Conformance to facial feature.

Breathing ports are covered with Viral/Bacterial filters.

Desiccant material is added to capture moisture accumulation.

Page 3 Face Plate

With a “normal” face mask, some straps are provided, extending from selected positions on the mask to features of the patient's face. Forces may be unevenly distributed about the periphery of the mask, which may allow for leakage.

In order to obtain a more air-tight seal between the mask and the patient's face, it is disclosed herein to use a separate faceplate to secure the mask to the patient's head (i.e., face). The faceplate is provided with its own straps, thereby negating the need for straps on the mask (although the mask straps may be left in place to allow first positioning the mask on the patient's face, then securing the mask to the face using the face plate/with its own straps.

The addition of a “face plate” serves to press the mask onto the facial features to improve fit.

The figure on the left illustrates that, in a conventional mask, there is leakage around nose due to stretch. A conventional mask has its own strap.

The figure on the right illustrates a faceplate which can be used with a conventional mask, or with some of the inventive mask embodiments disclosed herein. Note that the strap is relocated to the faceplate, and no longer extends from the mask itself. This provides a lot more control over fitting the mask securely to the patient's face, and may substantially reduce leakage from the mask.

Page 4

The addition of a “face plate” serves to press the mask onto the facial features to improve fit.

The figure on the left shows the face plate on a mask. The face plate distributes the strap force onto the facial features—nose, cheeks, and chin.

The figure on the right shows that the face plate is not fixed to the mask. It is adjusted to contact the patient's nose.

Page 5

This page illustrates various ways to optimize the Face Plate to provide:
1. Filter protection from user contact
2. Strap locking features
3. Nebulizer cutout

The face plate is sized and shaped to fit over the mask, a perimeter of the face plate being generally of the same size and shape as the mask. In use, the mask is retained between the face plate and the patient's face.

The face plate has its own straps (e.g. elastic bands), such as for securing the face plate to a user's head (i.e., face) by looping the straps over the patient's ears. This eliminates the need for straps on the mask.

The face plate may be made from a more rigid material than the mask. Because the face plate is relatively more rigid than the mask, forces exerted by the straps (such as elastic bands) may be more evenly distributed around the periphery of the face plate, hence around the corresponding periphery of the mask, to improve sealing of the mask, as well as providing greater comfort.

The FIG. (1) on the left shows that the face plate has a cutout on the bottom of the faceplate for easy mounting of a nebulizer device.

The FIG. (2) on the right shows strap locking features on the sides of the faceplate to provide enhanced holding pressure.

Page 6

This page shows some features for optimization of the mask ‘Face Plate’ to provide:
1. Filter protection from user contact
2. Strap locking features
3. Nebulizer cutout

The sole figure shows filter protection surfaces (1. Filter protection from user contact)

Page 7 and Page 8 of Appendix 4 are not related to faceplate features.

Appended hereto and forming a part of the specification hereof, is a 10 page document (Appendix B) illustrating and describing an adjustable face plate (or faceplate) for respiratory masks. The illustrations represent “other than line drawing” figures.

Page 1

This page shows the faceplate, such as described in US 20210330912 on a real world product, a large mask with nose and chin pads.

The illustration on the left is a front view, and the illustration on the right is a side view. These views illustrate that the vertical dimension (“H”) between a nose pressure point (“N”) located near the top of the faceplate and a chin pressure point (“C”) located near the bottom of the faceplate is fixed (non-adjustable). An exemplary dimension “H” may be 3.25 in (84 mm).

Page 2

This page shows that the non-adjustable faceplate of US 20210330912 does not fit all mask sizes. The two illustrations (left, right) show the non-adjustable faceplate of US 20210330912 fitted on a pediatric (small) mask, and illustrate the following problem with fitting a non-adjustable faceplate intended for a larger facemask over a smaller facemask.

The illustration on the left illustrates that there is a gap between the top portion of the faceplate and the top of the mask. This results, as also shown in the illustration on the right, that there is no nose pressure point. This defeats the purpose of the faceplate, which is to exert pressure on the top portion of the mask onto the patient's nose (bridge). (Of course, the faceplate has other intended purposes in addition to exerting pressure on the mask at the patient's nose.)

The fixed (adult) size faceplate of US 20210330912 is larger than the pediatric mask. As a result, the fixed (adult) size faceplate disclosed in US 20210330912 does not press the mask onto the pediatric patients nose.

A purpose of the faceplate is to press the mask onto the patient's face, all around the mask, helping to seal the mask to the patient's face.

One solution to the problem of the faceplate of US 20210330912 not fitting well with different size masks would be to have different size faceplates for use with different size masks. However, this would present the problem of stocking (in inventory) different size faceplates. A more desirable solution would be to have a “one size fits all” size faceplate.

Page 3

This page shows the contrast between the fixed (non-adjustable) faceplate of US 20210330912 (refer to the two illustrations on the left side of the page) versus the adjustable faceplate of the present invention (refer to the illustration on the right side of the page).

The illustrations on the left (one of which shows a decorative shield on the faceplate) show that there is a fixed distance between the nose pressure point and the chin pressure point, as discussed above.

The illustration on the right shows that the top portion of the faceplate has been modified to include a slider (discussed hereinbelow) that allow for a variable (adjustable) distance (“H”) between the nose pressure point and the chin pressure point. This feature allows the faceplate to apply appropriate pressure when used with different size facemasks.

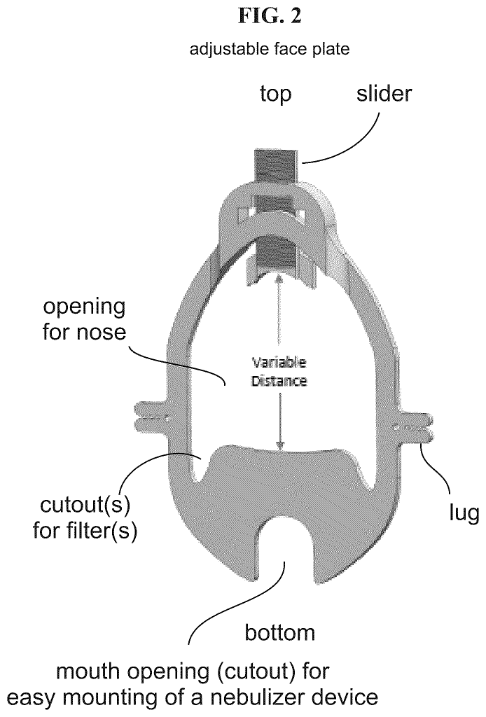

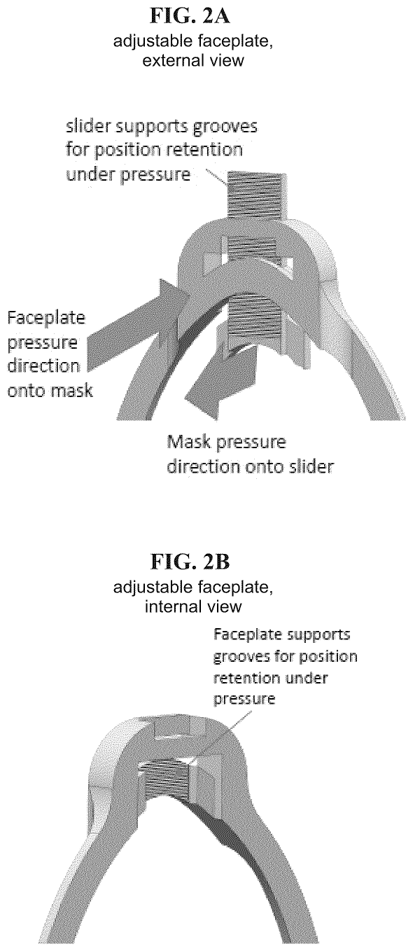

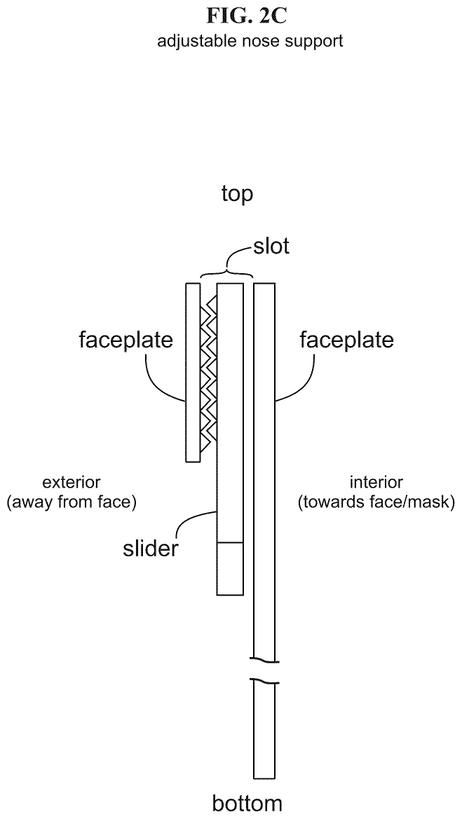

The illustration on the right side of Page 3 is reproduced herein as FIG. 2. The straps are omitted, for illustrative clarity.

Page 4

This page shows some features of the adjustable faceplate of the present invention.

The illustration on the left illustrates the outside (exterior) of the faceplate, the side of the faceplate away from the patient's face (away from the mask).

The illustration on the right illustrates the inside (interior, mask contact side) of the faceplate

In these, and some other illustrations, the mask is not shown (omitted), and the retaining straps for the faceplate are not shown (omitted), for illustrative clarity.

According to the invention, generally, a “slider” element is provided at the top (nose contour portion) of the faceplate, so that the distance (“H”) between the nose pressure point and the chin pressure point is adjustable, rather than fixed, so that the faceplate can effectively be utilized with different size masks (for different size patients).

The slider is disposed on (and retained by) the faceplate, at an upper (top) portion thereof in a manner that allows the slider to be moved up and down to respectively increase or decrease the distance (“H”) between the nose pressure point and the chin pressure point.

A bottom portion of the slider is contoured (shaped) to be compatible with the patient's nose, at which point on the patient's face the faceplate will be urging the mask against the patient's face.

The distance “F” (internal width of the faceplate) is dictated by the size of the mask, plus sufficient clearance for the “stick-on” filters typically 2.5 in (63 mm). (“F”=approximately 2.5 inches) The width of the opening is an additional feature designed to provide clearance for ‘stick-on’ filters. The dimension “F” (width of the nose opening) is sufficient to provide clearance to various types of filter attachments, not only stick-on filters.

Page 5

This page shows in greater detail how the slider is mounted to the faceplate, and some features thereof. The two illustrations on this page show only the upper (top) portion of the faceplate and the slider mounted thereto.

The illustration show that the slider is generally rectangular, having grooves on at least one surface thereof. The grooves in the slider may cooperate with a corresponding ridge or ridges on the faceplate so that the slider can easily be adjusted (up or down) and retained in the desired position when the faceplate is in use on a patient's face. Other means may be employed to allow for adjustment and retention, such as a friction (interference) fit.

The bottom of the slider is shaped to fit comfortably on the patient's nose (with the mask disposed between the bottom of the slider and the patient's face.

Page 6

This page shows the entire faceplate (without retaining straps).

The illustration on the left is an exterior view (away from the patient's face) and illustrates the slider moved (extended) to its bottommost position, such as for fitting to a small (child size) mask (mask not shown). Here, the distance between the nose pressure point and the chin pressure point is minimized. The slider may be considered to be fully extended in this illustration.

The illustration on the right is an interior view (towards the patient's face, or mask contact side of the faceplate) and illustrates the slider moved (extended) to its topmost position, such as for fitting to a large (adult size) mask (mask not shown). Here, the distance between the nose pressure point and the chin pressure point is maximized. The slider may be considered to be fully retracted in this illustration.

An exemplary amount of adjustment facilitated by the slider—in other words, the variation in the distance between the nose pressure point and the chin pressure point—may be approximately 1 inch (2.5 cm).

Page 7

This page shows some images of a prototype adjustable faceplate. Both images are exterior (away from the patient's face, away from the mask) views of the faceplate.

The illustration on the left illustrates the adjustable faceplate, with straps. In this image, the slider is in an intermediate (between topmost and bottommost) position, partially extended.

The illustration on the right illustrates that the slider has grooves (or ridges) for position retention under pressure.

Page 8

This page shows some images of a prototype adjustable faceplate. Both images are exterior (away from the patient's face, away from the mask) views of the faceplate.

The illustration on the left illustrates the adjustable faceplate prototype, with straps.

The illustration on the right illustrates that the upper portion of the faceplate, which supports the slider and allows the slider to move up and down, has grooves (or ridges) for cooperating with the corresponding grooves (or ridges) on the slider to retain the slider in its desired position, such as when the faceplate is in place over a facemask on a patient's face.

Page 9

This page shows the adjustable faceplate (face plate) of the present invention in conjunction with a pediatric/small size mask. The mask is shown supporting one filter over vent and one sealed vent, as may be described in US 20220347403.

The illustration on the left shows the faceplate with the slider at full extensions to make contact with a small (pediatric) mask.

The illustration on the right shows the faceplate with a nebulizer attached thereto.

Page 10

This page shows the adjustable faceplate of the present invention in conjunction with a medium size mask (left side illustration) and a large size mask (right side illustration). The mask is shown supporting dual filters over vents, as may be described in US 20220347403.

The illustration on the left shows the slider partially extended to make appropriate contact with a medium size mask.

The illustration on the right shows the slider fully retracted to make appropriate contact with a large size mask

### DRAWINGS AND DESCRIPTIONS

FIG. 1 is a diagram (plan view) of a face plate (or faceplate) for use with a face mask, such as was presented as FIG. 5 of US 20210330912. The faceplate is not adjustable, and represents “one size does not fit all.”

FIG. 2 is an illustration (plan view) of an adjustable faceplate, according to the invention. Compare Page 3 (right side illustration) of Appendix B.

FIG. 2A is a plan view of the exterior (side away from patient's face) of a top portion of the faceplate, illustrating the slider, according to the invention. Compare Pages 4, 5 and 6 (left side illustrations) of Appendix B.

FIG. 2B is a plan view of the interior (mask contact side) of a top portion of the faceplate, illustrating the slider, according to the invention. Compare Pages 4, 5 and 6 (right side illustrations) of Appendix B.

FIG. 2C is a cross-sectional view of a top portion of the faceplate, illustrating the slider, according to the invention.

In the adjustable faceplate shown in FIGS. 2, 2A, 2B (which may collectively be referred to as “FIG. 2”), a few things may readily be observed, in contrast with the non-adjustable faceplate shown in FIG. 1.

The overall shape of the FIG. 2 faceplate is similar to the overall shape of the FIG. 1 faceplate. The lugs for attaching the strap (not shown in the FIG. 2 adjustable faceplate) are slightly different than the lugs for attaching the strap in the FIG. 1 non-adjustable faceplate.

The top portion of the FIG. 2 adjustable faceplate is much different than the top portion of the FIG. 1 non-adjustable faceplate, and the differences will be described in greater detail hereinbelow.

The nose opening of the FIG. 2 adjustable faceplate is slightly different than the nose opening of the FIG. 1 non-adjustable faceplate. The FIG. 2 nose opening has side (left and right) portions extending downwards.

Page 3 of Appendix B shows, on the left side of the page, two illustrations of a “fixed” (non-adjustable) faceplate, of the prior art.

On the right side of the Page 3 of Appendix B is shown an adjustable faceplate, according to the present invention. See FIG. 2. In addition to being adjustable, the faceplate of the present invention is provided with cutouts at the bottom of the nose opening, on the left and right sides thereof, to permit filters, such as stick-on filters, to be mounted to a facemask underlying the faceplate. The cutouts are shown as extending downward from the nose opening, on either (i.e., the left and right) side of the faceplate. The cutout in the adjustable faceplate of the present invention for accommodating the stick-on filters, was not needed in the US 20210330912 non-adjustable faceplate since the filters were welded to the mask and do not protrude from the mask.

Both of the above illustrations on Page 3 of Appendix B show that the bottom cutout (for mounting a nebulizer device) may be U-shaped, rather than a circular opening (as was shown in FIG. 1).

The illustration on the left side of page 9 of Appendix B shows a pediatric mask with the faceplate slider at full extension for a mask with as single stick-on filter on one vent hole and a sealed cover on second vent hole.

The opening (cutout) in the lower portion of the FIG. 2 adjustable faceplate of the present invention is different than the opening in the lower portion of the FIG. 1 non-adjustable faceplate of US 20210330912. Rather than the opening in the lower portion of the non-adjustable faceplate being fully enclosed about its circumference, the opening (cutout) in the lower portion of the adjustable faceplate of the present invention is open at the bottom. This feature facilitates easy mounting of a nebulizer device to the faceplate, and is independent of the adjustable feature, which will be described in greater detail hereinbelow.

The FIG. 2 adjustable faceplate of the present invention may be made mostly from the same or similar material as the FIG. 1 non-adjustable faceplate of US 20210330912, for example, 2.0-3.0 mm (0.080″-0.125″) thick PVC (plastic).

Adjustable Nose Support

At a glance, it can readily be noticed that top portion of the FIG. 2 adjustable faceplate is provided with an adjustable nose support element comprising:
a moveable element (or “slider”), extending from a top portion of the faceplate into the nose opening of the faceplate; and
features on the top portion of the faceplate for grasping the slider in a manner that it may be moved up and down to accommodate patients having different size faces.

The non-adjustable faceplate as described in US 20210330912 is not provided with an adjustable nose support feature.

The slider, in cooperation/conjunction with appropriate modifications to the top portion of the faceplate (slot for receiving the slider, etc.), allows the spacing (distance “H” between the nose pressure point and the chin pressure point) of the adjustable faceplate to be modified according to the size of the patient's face (and facemask being used), so that the adjustable faceplate can exert appropriate pressure upon the face mask (not shown) to ensure (improve) sealing of the facemask on the patient's face. Refer to Pages 3, 4 and 5 of Appendix B. In other words, the effective vertical dimension (“H”) of the adjustable faceplate can be adjusted to accommodate an adult (large), a child (medium), or an infant (small) face and facemask. Generally, this feature (function) is accomplished by allowing the slider to be moved up or down to a desired position then, when securing (with the straps) the faceplate to the patient's head, pressure is exerted on the slider which will “lock” the slider in place on the faceplate, establishing the desired distance “H” between the nose and chin pressure points.

FIG. 2A corresponds with the illustration on the left side of Page 5 of Appendix B, and illustrates the slider which is movable (up and down) in a structure on the top portion of the adjustable faceplate. this is an external view, from the side of the adjustable faceplate, away from the patient's face.

FIG. 2B corresponds with the illustration on the right side of Page 5 of Appendix B, and illustrates the slider which is movable (up and down) in a structure on the top portion of the adjustable faceplate. This is an external view, from the mask contact side of the adjustable faceplate, towards the patient's face.

The slider is generally rectangular, having a length a few (such as 3 or 4) times greater than its width. The slider is shown as being provided with grooves or ridges on a surface thereof, such as the front (exterior, away from the patient's face) side of the slider. A bottom portion of the slider may be wider than the main body portion of the slider, and is shown as being contoured to adapt to the patient's nose (bridge).

The top portion of the faceplate is provided with a slot, within which the slider may be retained in the top portion of the faceplate while allowing the slider to be positioned up or down, or “moveably supported” (up or down) within the slot for adjusting the size of the nose opening depending on the size of the patient's face. See Page 3 of Appendix B which contrasts the variable distance for the adjustable faceplate with the fixed distance for the non-adjustable faceplate described in US 20210330912.

A portion of the slot at the top portion of the faceplate is shown as being provided with grooves or ridges on a surface thereof, such as an interior (towards patient's face) side of the slot.

FIG. 2C is a cross-sectional view of the slider and the slot on the top portion of the adjustable faceplate. In order to accommodate, and “capture” the slider, the top portion of the faceplate may be thicker than remaining portions of the faceplate.

The slider is shown as being provided with grooves or ridges (i.e., features forming a non-smooth surface) on the front (exterior, away from the patient's face) side (surface) of the slider, and the faceplate is shown having corresponding non-smooth surface features such as grooves or ridges on the corresponding (facing the exterior surface of the slider) portion of the faceplate forming the slot and interacting with the slider. Alternatively or additionally, similar non-smooth surface features such as grooves or ridges may be formed on the interior surface of the slider and the corresponding (facing the interior surface of the slider) portion of the faceplate forming the slot.

Methods other than ridges and grooves could be used to secure the slider at the required position such as a threaded screw or clamp.

In use, the faceplate is urged against the facemask (not shown) on the patient's face by the strap(s) (not shown, see FIG. 1)

The leftmost illustration on Page 3 of Appendix B shows a fixed (non-adjustable) faceplate with the addition of a element which may be referred to as a “bridge” or a “decorative shield” extending generally horizontally across the faceplate, from left-hand side to right-hand side thereof, approximately midway between the top and bottom thereof.

According to a feature of the invention, the additional “bridge” or a “decorative shield” may be integrally formed with the faceplate. The purpose(s) of the decorative shield may be to enhance rigidity of the faceplate, protect filters mounted to the facemask from contact (contamination), to make faceplate more friendly for kids (masks with characters on them exist for that purpose), to add grasping supports, or to enhance perceived value, including combinations thereof. The fixed (non-adjustable) faceplate, without the “bridge” or a “decorative shield” is prior art.

Some Additional Comments and Features

The face plate is not fixed to the mask. It is adjusted to contact the patient's nose (and chin).

The face plate may push the mask against a wearer's facial features.

A strap may be disposed on the face plate, rather than on the mask.

The face plate distributes the strap force onto the facial features—nose, cheeks, and chin

The face plate may provide or comprise:
1. Filter protection from user contact
2. Strap locking features
3. Nebulizer cutout

A cutout may be provided on the bottom of the faceplate, such as for easy mounting of a nebulizer device.

A cutout may be provided in the nose opening of the faceplate to accommodate the filter shape.

Strap locking features may be provided on the sides of the faceplate to provide enhanced holding pressure, and adjustability.

Filter protection surfaces may be provided on the face plate.

The faceplate may provided with a nose cushion to increase conformance to the patient's facial feature.

While the invention(s) may have been described with respect to a limited number of embodiments, these should not be construed as limitations on the scope of the invention(s), but rather as examples of some of the embodiments of the invention(s). Those skilled in the art may envision other possible variations, modifications, and implementations that are also within the scope of the invention(s), and claims, based on the disclosure(s) set forth herein.

What is claimed is:

1. An adjustable faceplate for securing a respiratory facemask to the face of a patient, comprising:
a structure having an opening having a height “H” and a width “F”, and a peripheral portion conforming to a corresponding peripheral portion of the facemask, and suitable to be disposed over the facemask when the facemask is on the patient's face; and
at least one strap extending from the structure for securing the faceplate to the patient's face, thereby negating a need for a straps extending from the facemask;
a slider disposed at a top portion of the faceplate for providing a nose pressure point, wherein the a distance between the nose pressure point and a chin pressure point at a bottom portion of the faceplate is adjustable by moving the slider so that the faceplate can perform well with masks of different sizes for adult, child, and pediatric patients.

2. The adjustable faceplate of claim 1, wherein:
the structure is formed of a material that is more rigid than the material of the facemask.

3. The adjustable faceplate of claim 2, wherein:
the structure spreads forces from the strap(s) more evenly around the peripheral portion of the facemask, thereby enhancing a seal between the facemask and the patient's face.

4. The adjustable faceplate of claim 1, wherein:
the structure has a cutout disposed at a bottom portion thereof to accommodate mounting a nebulizer device.

5. The adjustable faceplate of claim 1, wherein:
the structure of sufficient width to accommodate filters attached to facemask.

6. An adjustable faceplate, for use with respiratory masks, comprising:
an adjustable nose support element comprising:
a moveable element, extending from a top portion of the faceplate into a nose opening of the faceplate; and
features on a top portion of the faceplate for grasping the slider in a manner that it may be moved up and down to accommodate patients having different size faces.

7. The adjustable faceplate of claim 6, wherein:
the slider is provided with grooves or ridges on a surface thereof; and
the features on the top portion of the faceplate comprise grooves or ridges for mating with the grooves or ridges on the slider.

8. The adjustable faceplate of claim 7, wherein:
the grooves or ridges on the slider and the top portion of the faceplate allow the slider to be moved up or down to accommodate masks of different sizes and to retain the slider in place when the faceplate is installed on the patient.

9. The adjustable faceplate of claim 6, wherein:
the slider allows a distance between a nose pressure point and a chin pressure point of the adjustable faceplate to be modified according to the size of the patient's face and facemask being used, so that the adjustable faceplate can exert appropriate pressure upon the facemask to improve sealing of the facemask on the patient's face.

10. The adjustable faceplate of claim 6, wherein:
in use, the faceplate urges the facemask against a wearer's facial features, particularly at a nose pressure point and a chin pressure point.

11. A method of mounting an adjustable facemask onto the face of a patient comprising:
applying a medical facemask onto the patient's face; and
securing the facemask in place on the patient's face with a faceplate.

12. The method of claim 11, wherein:
the faceplate includes a strap.

13. The method of claim 11, wherein:
adjusting the height of a nose opening in the faceplate to accommodate different size facemasks.

14. The method of claim 11, wherein:
the faceplate is provided with cutouts extending from the nose opening to accommodate filters attached to the facemask.

15. The method of claim 11, further comprising:
mounting a nebulizer to the faceplate.
